(19) United States
(12) Patent Application Publication
(10) Pub. No.: US 2026/0213026 A1
(43) Pub. Date: Jul. 23, 2026

(54) METHODS AND SYSTEMS FOR OPTIMIZING COMMUNICATIONS BASED ON COMMUNICATION ATTRIBUTES

(71) Applicant: Sequelae, Inc., Minneapolis, MN (US)

(72) Inventors: James LaBelle, Encinitas, CA (US); Shane Hoversten, Brooklyn Park, MN (US); Benjamin C. Wiegand, Yardley, PA (US); Daniel J. Kunnath, JR., Ann Arbor, MI (US); Steven J. Catani, Athens, GA (US)

(73) Assignee: Sequelae, Inc., Minneapolis, MN (US)

(21) Appl. No.: 19/450,861

(22) Filed: Jan. 16, 2026

Related U.S. Application Data
(60) Provisional application No. 63/747,579, filed on Jan. 21, 2025.

(51) Int. Cl. G16H 80/00; G16H 15/00; G16H 20/00; G16H 50/20; G16H 50/30

(52) U.S. Cl. CPC G16H 80/00; G16H 15/00; G16H 20/00; G16H 50/20; G16H 50/30

(57) ABSTRACT

Methods and systems for optimizing communications based on communication attributes are provided. A digital communication network (DCN) is provided to a plurality of members of a population to communicate with each other and the DCN. An individual member is identified from the plurality of members in the DCN and a target communication attribute associated with the individual member is determined. For each communication, an associated level of the target communication attribute is determined and an order of the plurality of communications is prioritized based on the associated level of the target communication attribute.

### CROSS REFERENCE TO RELATED APPLICATIONS

This application claims the benefit of U.S. Provisional Application No. 63/747,579, filed on Jan. 21, 2025, which application is incorporated herein by reference in its entirety.

### BACKGROUND

Various embodiments relate generally to health care systems, methods, devices and computer programs and, more specifically, relate to promoting healthy lifestyles based on attributes of communications in a digital communication network.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

Computers have changed the way people interact. Digital communication networks, which can include social media where individuals can interact with others online, connect many with a community. Computers can also interact with people using sophisticated programs. These programs can mimic human interaction and provide information to people. The online interactions, whether through a community or a computer bot, can be utilized to help build positive behaviors and encourage people to make improvements in their lives.

People often take actions—whether simple, hard, good, or bad, etc.—that impact their health. Regardless, such actions have consequences that can accumulate over time. However, rarely do people associate such actions with consequences. This may be due to, for example, the lack of data to see the consequence (or the data is available in a way that does not connect to the action). Other times, they may not think about the connection between the action and the consequence. Reflecting on their conditions can help people associate actions with their consequences; thus, it is desirable for people to reflect on their actions and potential consequences.

Mindfulness, as gained through reflection, is an essential part of self-improvement. Unless someone is aware of their behaviors and how those behaviors impact the things that are important to them, it is very hard to sustain change in their behaviors. Reflection is the process by which people gain that understanding. Reflecting takes conscience effort and is often emotionally draining and cognitively draining. Thus, a person may find it easier to not reflect on such actions.

Computers have changed the way people interact. Digital networks, which may include the use of social media, allow individuals to interact with others online and connect many people with a community. The online community can be utilized to help build positive behaviors and encourage people to make improvements in their lives. Computers can also provide a way for people to record their actions and review them later as well as to enable a person to reflect on their activities

What is needed is a way to promote positive lifestyles through healthy engagement in a digital communication network.

### SUMMARY

Example aspects of the present disclosure include:

A method according to at least one embodiment of the present disclosure comprises providing a digital communication network (DCN) to a plurality of members of a population to communicate with each other and the DCN; identifying an individual member from the plurality of members in the DCN; determining a target communication attribute associated with the individual member, wherein the target communication attribute corresponds to a communication attribute valued by the individual member; analyzing a plurality of communications from the plurality of members to determine, for each communication, an associated level of the target communication attribute; and prioritizing an order of the plurality of communications based on the associated level of the target communication attribute, wherein communications with higher associate levels of the target communication attribute are prioritized higher than communications with lower or no associated levels of the target communication attribute.

Any of the aspects herein, wherein the target communication attribute comprises one or more of: empathy, attachment, introspection, and agency.

Any of the aspects herein, wherein the plurality of communications are a first plurality of communications analyzed during a first period of time and the associated level of the target communication attribute is a first associated level of the target communication attribute, and wherein the method further comprises: generating a first target communication to the individual member based on the target communication attribute; transmitting the first target communication to the individual member during a second period of time, the second period of time occurring after the first period of time; analyzing a second plurality of communications during the second period of time to determine a second associated level of the target communication attribute; generating a second target communication to the individual member based on a difference between the first associated level of the target communication attribute and the second associated level of the target communication attribute; and transmitting the second target communication to the individual member.

Any of the aspects herein, wherein the first period of time and the second period of time are one of: one or more days, one or more weeks, and one or more months.

Any of the aspects herein, wherein the first target communication includes a plurality of summaries, each summary describing a subset of communications from the plurality of communications that are similar to each other, and wherein the second target communication includes a modified plurality of summaries.

Any of the aspects herein, wherein different summaries of the plurality of summaries are transmitted to different members of the plurality of members.

Any of the aspects herein, wherein the first target communication includes a first recommendation for a tool associated with living a healthy lifestyle and the second target communication includes a second recommendation for an adjustment to the tool or a different tool.

Any of the aspects herein, wherein the tool comprises at least one of a wearable device configured to measure a biomarker, a kit to measure a biomarker, a health-inducing game, a psychometric instrument, and a health-improving program.

Any of the aspects herein, wherein the method is used to slow progression of an adverse health condition of the individual member.

Any of the aspects herein, wherein the adverse condition is determined by automatically analyzing communications from the individual member in the DCN.

Any of the aspects herein, wherein the adverse condition comprises at least one of chronic systemic inflammation, malaise, low energy, a disease, a health risk, social dysfunction, or a prodromal disease.

Any of the aspects herein, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

Any of the aspects herein, wherein the health risk is at least one of falling or becoming infected with an illness.

A method according to at least one embodiment of the present disclosure comprises providing a digital communication network (DCN) to a plurality of members of a population to communicate with each other and the DCN; identifying an individual member from the plurality of members in the DCN; determining a target communication attribute associated with the individual member, wherein the target communication attribute corresponds to a communication attribute valued by the individual member; analyzing a first plurality of communications from the plurality of members over a first period of time to determine: a first degree to which a set of actions were taken by the plurality of members during the first period of time, and a first associated level of the target communication attribute; analyzing a second plurality of communications from the plurality of members over a second period of time to determine: a second degree to which the set of actions were taken by the plurality of members during the second period of time, and a second associated level of the target communication attribute; prioritizing an order of the subset of communications based on the first associated level of the target communication attribute, the second associated level of the target communication attribute, the first degree, and the second degree.

Any of the aspects herein, wherein the order of the subset of communications is further prioritized to increase a probability of adoption by the individual member of an action from the set of actions.

Any of the aspects herein, wherein the order of the subset of communications is further prioritized to increase a probability of the individual member maintaining the adopted action.

Any of the aspects herein, wherein the set of actions comprises at least one of: lifestyle changes, medical interventions, changes in social interactions, and changes in a living environment.

Any of the aspects herein, wherein a difference between the first degree and the second degree meeting the predetermined threshold correlates to at least one of an increased degree or a decreased degree of the set of actions taken by the plurality of members.

Any of the aspects herein, wherein the method is used to slow progression of an adverse health condition of the individual member.

A system according to at least one embodiment of the present disclosure comprises a computer processor; a data repository in communication with the computer processor and storing: a plurality of communications, a communication attribute, and an associated level of a communication attribute; a digital communications network (DCN) which, when executed by the computer processor, provides a network for a plurality of members of a population to interact with at least each other or the DCN; and a server controller which, when executed by the computer processor: identifies an individual member from the plurality of members in the DCN; determining a target communication attribute associated with the individual member, wherein the target communication attribute corresponds to a communication attribute valued by the individual member; identifies a plurality of communications from the plurality of members; analyzes the plurality of communications from the plurality of members to determine, for each communication, an associated level of the target communication attribute; and prioritizes an order of the plurality of communications based on the associated level of the target communication attribute, wherein communications with higher associate levels of the target communication attribute are prioritized higher than communications with lower or no associated levels of the target communication attribute.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3 is a logic flow diagram that illustrates the operation of a method, in accordance with one or more embodiments.

FIG. 4 is a logic flow diagram that illustrates the operation of a method, in accordance with one or more embodiments.

FIG. 5A shows an example of a computing system, in accordance with one or more embodiments.

FIG. 5B shows an example of a network, in accordance with one or more embodiments.

### DETAILED DESCRIPTION

Digital communications networks, especially the subset known as social networks, are typically financed through a business model based on advertising. As such, conventional social networks focus on getting members to engage with the network as frequently and deeply as possible in order to draw more members into their fold through prioritizing messages in a members communication feed designed to maintain engagement. Such prioritization may be based on tribalism, anger and/or fear.

Various embodiments of the present disclosure use a digital communication network (DCN) to promote health. The DCN is intended to care about positive engagement to encourage members to take positive actions to improve their health. These improvements can be lifestyle changes, adoption and compliance with medical interventions, activities that induce a positive emotional state and the like. Members both give and receive the benefits of ideas and support through communication on the DCN. Through this process the members build self-efficacy and agency, the belief that they can impact their own outcomes through their actions, and this agency extends to other aspects of their lives beyond their health, increasing overall wellbeing.

Like conventional systems, the DCN can optimize for engagement. However, rather than focus on engagement alone, without consideration of the repercussions of the potential negative emotions, the DCN may instead spread “action” and generate agency messages which have certain attributes. Four such attributes are empathy, attachment, introspection, and self-efficacy. The DCN can derive these attributes from analysis of messages, and optimizes for action out of the network in part by prioritizing messages that have the type of attributes which drive this outcome.

Empathy, attachment, introspection, and agency are attributes of communications that can contribute to the efficacy of a DCN in spreading ideas as medicine. They are different from (and in some ways at odds with) standard engagement which is driven by advertising-based models. As with other attributes, the DCN may use AI bots trained on sample data, such as test results, in order to assign a ranking for the attribute to a message. The DCN may also use such AI bots to generate messages which intended to reflect (or induce) such attributes, for example, by being ranked highly in one or more such attributes.

The DCN can analyze individual messages to gauge the level of the communication attributes. Alternatively, the DCN may analyze multiple messages in a conversation in order to assign an associated level of the communication attributes to the conversation (either in part or in whole). The DCN can also evaluate conversations over multiple periods of time in order detect changes in the communication attributes, for example, to compare the level of agency from a first month to the level of agency from a second, subsequent month.

The generated messages may be used to communicate information regarding various lifestyle options, such as interventions, health-inducing tools available to the population, opportunities to engage in reflection, etc. Some generated message can provide the knowledge directly, direct the user to a tool to help them achieve healthier lifestyles and/or communicate information regarding the various tools available.

The individual messages may be relayed separately, for example, in an ordered sequence. The order messages are provided may be arranged based on the rankings for various attributes, such as those messages having higher empathy rankings being shown before those with lower empathy rankings. Likewise, some message may be omitted if the desired attribute ranking does not reach a given threshold.

The DCN may also generate summaries of the multiple communications. Similar types of communications may be combined and analyzed to create the summaries. The summaries may include copies of the communications, directions to access the communications and/or snippets of the communications. The order of the communications in the summaries may be based on the rankings of the attributes for each communication.

User engagement may be used by the DCN to identify better information for the user, for example, to locate more applicable community or expert-sourced answers for the user. The DCN may also increase user response by tailoring messages for user engagement. As the user engagement is based on healthy attributes, such as empathy, the user may associate positive emotions with the messages and topics discussed and may be provided to care givers and/or physicians in order to better evaluate the user.

Such engagement may be used to create greater awareness of lifestyle changes and medial options. A lifestyle (or behavior) change can be evaluated based on the user's engagement. The message attributes may also be used to help identify the user's condition, for example, to evaluate whether a lifestyle change is working.

Additionally, the user's engagement can be used to provide the user's experience as a social aspect. A social value can be placed on those members of a community that have relevant experience. The engagement from the users can be induced in order to help elevate the aspects of the experience that may be more instructional to the community as a whole. This can help increase the number of members of the community thinking about an action, for example, creating an atmosphere where people can learn about others in who have undergone a change they are considering. Users who have positive engagement with a message may be well situated to create wide-range engagement in the community.

Additionally, the community can be used to help support the patient in other ways. Lifestyle interventions are inherently safe. They use the philosophy that any action now is preferred to a “better” action later. They also support the concept that ideas and communication are healthcare.

Further, Lifestyle, environmental, and social group interventions have two characteristics different from other health related interventions. First, they have even more person-to-person variance than drug or surgical interventions, making creating a decision scheme to select the best one in a “feed forward way” even more difficult. Second, they have very low cost and risk. This should indicate that the decision process should shift from feed forward to “feedback” where you try something and then use the biometric to see if it worked. Of course, “feed forward” logic can be used to suggest things to try, but medical practitioners can also use intuition, preference, and what they have learned from previous attempts to guide the choice.

Despite this unquestionable logic, the health system sticks to “feed forward”. It's what they are used to, it's what the FDA and clinical study complex knows how to do, and it allow direct, but flawed comparisons to medical interventions. Mostly though, feedback takes time and a provider system based on selling a physician's time doesn't make the types of interactions required to iterate in feedback way practical.

This can be overcome in multiple ways. Inducing user engagement (by promoting communications with high levels of target communication attribute) can improve member usage of healthy activities without increasing healthcare system costs. This can also be used to improve community awareness of interventions. In some situations, biometric tools can also be provided directly to the users to help assess if their actions are working.

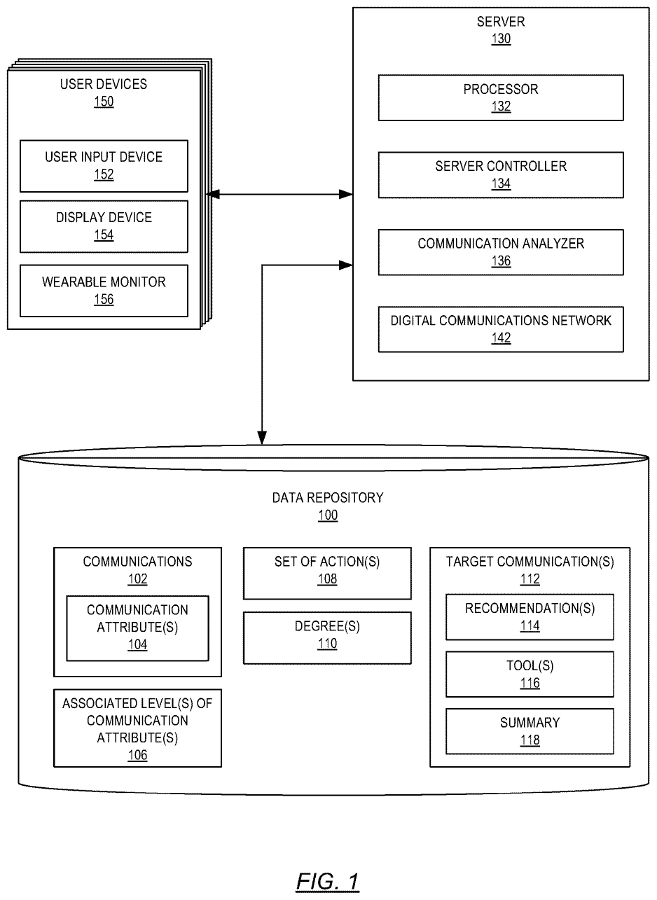

Turning to the Figures, the system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communications (102). The communications (102) are communications from members in a digital communication network (DCN) (142) (described in more detail below). The communications (102) may be, for example, text-based, image-based, multimedia communications, audio communications, or any combinations thereof. The communications may be between members, communications in a forum, communications with the DCN (142), or communications generated by the DCN (142) (e.g., communications from a conversation bot, also referred to as a chat bot). The communications (102) may include, for example, a post, a direct message, a comment, and/or visual and/or audio media by one or more members of the DCN (142) or generated by the DCN (142) (or a server controller such as a server controller (134) of the DCN (142)). The communications (102) may also be referred to as bot communications when the communications are generated and received from one or more conversation bots of the DCN (142).

The communications (102) in the DCN (142) include one or more communication attributes (104). The communication attributes (104) include, for example, empathy, attachment, introspection, and self-efficacy. The communication attributes (104) can be determined or derived from communications (102) by, for example, analyzing the communications (102) using a communication analyzer such as the communication analyzer (136).

The communication attributes (104) can be used to encourage, optimize, or increase activity by one or more members of the plurality of members in order to, for example, reduce an adverse health risk or increase a healthy lifestyle of the one or more members. Such optimization can include, for example, prioritizing communications that have the communication attributes valued by the member(s) such that the member(s) are more receptive to the prioritized communications. In another example, target communications such as target communications (112) (described below), can be generated based on one or more target communication attributes.

The data repository (100) also stores associated level(s) of communication attribute(s) (106) (also referred to as associated level(s) (106)). The associated level (106) is a level of a communication attribute (104) in a communication (102). In other words, the associated level (106) measures an amount or degree of the communication attribute (104) in the communication (102). Thus, communications (102) with a higher associated level (106) exhibits more of the communication attribute (106) than communications (102) with a lower associated level (106) of the communication attribute (106). For example, a first communication may have a higher level of introspection than a second communication. In such example, a member who prioritizes introspection may be more receptive to the first communication compared to the second communication.

The data repository (100) also stores set(s) of action(s) (108). The set of actions (108) are actions that can be taken by the members of the DCN (142). The set of actions (108) can include lifestyle changes, medical interventions, changes in social interactions, and changes in a living environment. For example, the set of actions (108) can include yoga, a prescription, hiking, jogging, in-person meeting versus online meeting, etc.

The data repository (100) also stores degree(s) (110). The degrees (110) correspond to how much a member has participated in or performed the action(s) (108). For example, a member who participates in yoga weekly will have a higher degree (110) of the action (108) yoga than a member who participates in yoga once a month. The degree (110) can be a numerical value or a percentage.

The data repository (100) also stores target communication(s) (112). The target communications (112) are communications directed to an individual member of the plurality of members and are generated by the server controller (134) of the DCN (142) for the individual member. The target communication (112) may be generated based on a member's target communication attribute. For example, the target communication (112) may include words of encouragement for a member who's target communication attribute is self-efficacy.

In some embodiments, the target communication (112) may include a recommendation (114). The recommendation (114) may include recommendations for the member to perform one or more of the set of actions (108). In another example, the recommendation (114) may recommend a tool (116) for the member to use. The tool (116) may be, for example, associated with living a healthy lifestyle and can include a wearable device configured to measure a biomarker, a kit to measure a biomarker, a health-inducing game, a psychometric instrument, or a health-improving program. Such tools (116) can be used to track or monitor the member's health or an adverse health condition. Such tools (116) can also be used to determine if actions (108) taken by the member have improved their health or adverse health condition.

In other embodiments, the target communication (112) may include a summary (118) of communications (102) from other members of the DCN (142). The summary (118) may describe a subset of communications (102) from a plurality of communications that are similar to each other. The subset of communications (102) may also have higher associated levels of a communication attribute (106) than communications (102) not included in the subset of communications (102). A style or words used in the summary (118) may be based on the communication attribute (104) with the higher associated levels (106).

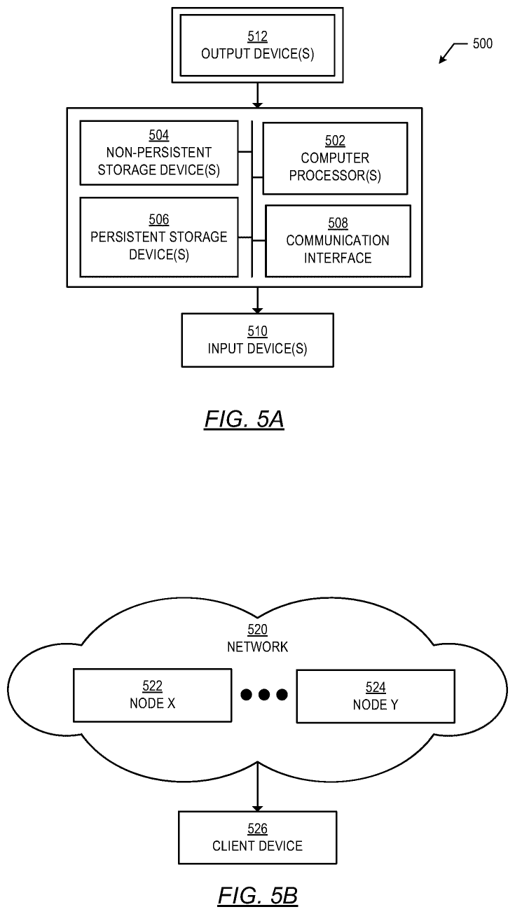

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136). An example of a computer system and network that may form the server (130) is described with respect to FIG. 5A and FIG. 5B.

The server (130) also includes a computer processor (132). The computer processor (132) is one or more hardware or virtual processors which may execute computer readable program code that defines one or more applications, such as the communication analyzer (136). An example of the computer processor (132) is described with respect to the computer processor(s) (502) of FIG. 5A.

The server (130) also may include a server controller (134). The server controller (134) is software or application specific hardware which, when executed by the computer processor (136), controls and coordinates operation of the software or application specific hardware described herein. Thus, the server controller (134) may control and coordinate execution of the communication analyzer (136).

The server (130) also includes the communication analyzer (136). The communication analyzer (136) is software or application specific hardware which, when executed by the computer processor (132), analyzes communication (102) in the DCN (142), determines the associated levels of communication attributes (106) of each communication (102) and/or degree(s) (110) to which members of the DCN (142) have taken sets of actions (108) based on the analysis, and generates the target communications (112). In other instances, the communication analyzer (136) analyzes communication (102) in the DCN (142), determines the associated levels of communication attributes (106) of each communication (102) based on the analysis, and prioritizes an order of communications (102) to present to an individual member based on the associated levels of communication attributes (106).

The server (130) also includes a digital communications network (DCN) (142). The DCN (142) is a network through which members of a population can interact with each other, or with a system supported by the DCN (142). The DCN (142) can also interact with the members of the population and, for example, transmit communications (102) and target communications (112) to one or more members of the DCN (142).

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (500) shown in FIG. 5A) that communicate with the server (130). The user devices (150) may include a wearable monitor (156). In an alternative embodiment, a separate wearable device may be in communication with the user device (150), such as a smart watch, or blood pressure monitor. The user devices (150) may also include a user input device (152) and/or a display device (154).

While FIG. 1 shows a configuration of components, other configurations may be used without departing from the scope of one or more embodiments. For example, various components may be combined to create a single component. As another example, the functionality performed by a single component may be performed by two or more components.

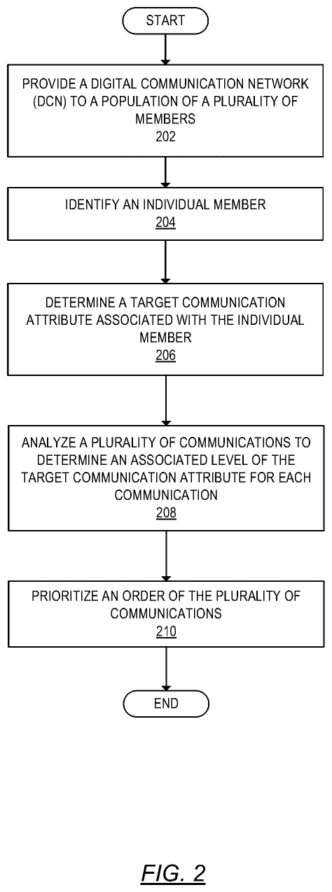

FIG. 2 is a logic flow diagram that illustrates a method, and a result of execution of computer program instructions, in accordance with various embodiments. The method can be used to prioritize an order of communications based on a target communication attribute of an individual member. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 202, a step of providing a digital communication network (DCN) to a population is provided. The DCN may be the same as or similar to the DCN (142). As previously described, members of the population enrolled in the DCN can communicate with each other or with the DCN itself. The members of the population can also communication with one or more conversation bots.

At Block 204, a step of identifying an individual member is provided. The individual member may be identified via one or more communications by the individual member. The communication may be the same as or similar to the communication (102). In some embodiments, the communications may be a post by the individual member in the DCN.

At Block 206, a step of determining a target communication attribute associated with the individual member is provided. In some embodiments, communications from the individual member can include a target communication attribute such as the communication attribute (104). The target communication attribute may correlate to a communication attribute that the individual member values. Thus, the target communication attribute may indicate that the individual member may value or be more receptive to a communication with the target communication attribute. In other embodiments, the target communication attribute may be provided by the individual member via, for example, a user device such as the user device (150).

In some embodiments, the Block 206 may be an optional step. In such embodiments, the target communication attribute may be automatically selected by, for example, the DCN. For example, the DCN may determine the associated levels of communication attributes for each communication and the DCN may select the communication attribute with the highest associated level as the target communication attribute.

At Block 208, a step of analyzing a plurality of communications to determine an associated level of the target communication attribute for each communication is provided. The plurality of communications includes communications such as the communications (102) and the associated level of the target communication attribute may be the same as or similar to the associated level of communication attributes (106). Analyzing the plurality of communications can include using the plurality of communications as input to a communication analyzer such as the communication analyzer (136) and receiving the associated levels of the communication attributes for each communication. Each communication can have one or more communication attributes and thus, each communication can also have one or more associated levels.

At Block 210, a step of prioritizing an order of the plurality of communications is provided. The order of the plurality of communications may be prioritized based on the associated levels of each communication. By prioritizing communications with higher associated levels of the target communication attribute of the individual member, the individual member may be more receptive to the prioritized communications that exhibit higher levels of the target communication attribute. The communications with higher levels of the target communication attribute may also encourage the individual member to practice such target communication attributes.

The method described in FIG. 2 can include more or less steps. One or more steps or any combination of steps may also be repeated in the method described in FIG. 2.

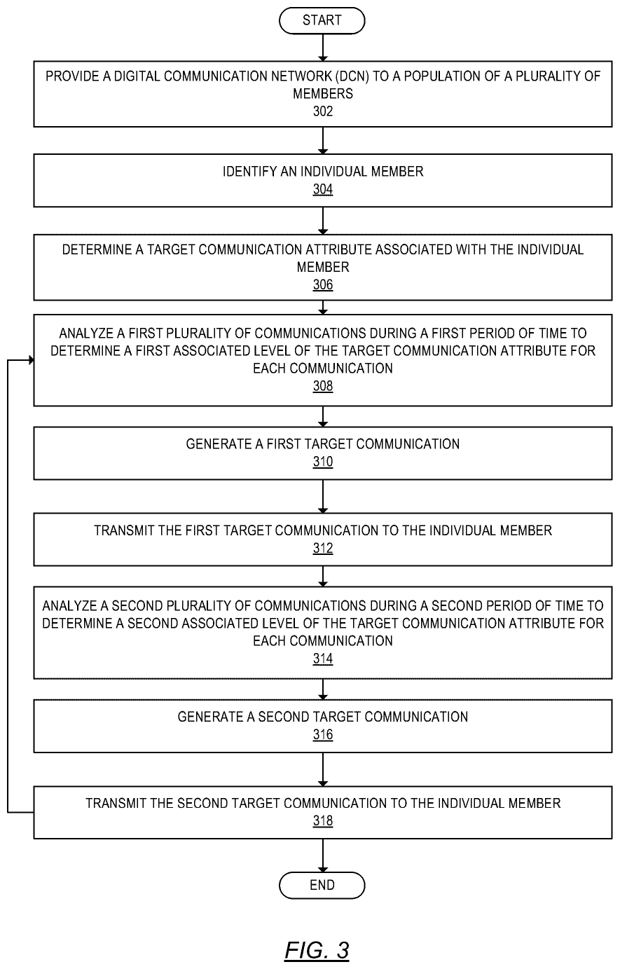

FIG. 3 is a logic flow diagram that illustrates a method, and a result of execution of computer program instructions, in accordance with various embodiments. The method can be used to generate target communications for an individual member based on the individual member's target communication attribute. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 302, a step of providing a digital communication network (DCN) to a population is provided. The step of Block 302 may be the same as or similar to the step of Block 202 of FIG. 2 described above.

At Block 304, a step of identifying an individual member is provided. The step of Block 304 may be the same as or similar to the step of Block 204 of FIG. 2 described above.

At Block 306, a step of determining a target communication attribute associated with the individual member is provided. The step of Block 306 may be the same as or similar to the step of Block 206 of FIG. 2 described above.

At Block 308, a step of analyzing a first plurality of communications during a first period of time to determine a first associated level of the target communication attribute for each communication is provided. The step of Block 308 is the same as or similar to the step of Block 208 of FIG. 2, described above, except that the plurality of communications spans the first period of time. The first period of time may be, for example, one or more hours, one or more days, one or more weeks, and one or more months. In other instances, the first period of time may span any time period.

At Block 310, a step of generating a first target communication is provided. The first target communication may be the same as or similar to the target communication (112). The first target communication can be based on the individual member's target communication attribute and communications that have high associated levels of the target communication attribute. The first target communication can be based on, for example, a style or wording of communications that have high associated levels of the target communication attribute. In other examples, the first target communication may include some of the communications with high associated levels of the target communication attribute.

As previously described, the target communication can include a recommendation such as the recommendation (114) and/or a summary such as the summary (118) of a subset of communications from other members in the DCN. The recommendation can also include a recommendation for the individual member to use a tool such as the tool (116) to improve or monitor their health. The summary may be useful to the individual member as members may value communications from other members or peers.

At Block 312, a step of transmitting the first target communication to the individual member is provided. The first target communication may be transmitted to the individual member via a user device such as the user device (150).

At Block 314, a step of analyzing a second plurality of communications during a second period of time to determine a second associated level of the target communication attribute for each communication is provided. The step of Block 314 is the same as or similar to the step of Block 308, except that the second period of time occurs after the first period of time begins.

At Block 316, a step of generating a second target communication is provided. Generally, the step of Block 316 is the same as or similar to the step of Block 310. The second target communication differs from the first target communication in that the second target communication may be a modified first target communication based on a difference between the first associated level of the target communication attribute and the second associated level of the target communication attribute.

For example, the first target communication may include a recommendation for the individual member to use a tool used by other members that generated the plurality of communications. In the same example, the difference may indicate that the associated levels of the target communication attribute of the plurality of communications has decreased, and thus, the plurality of communications from the second period of time may not be as effective to the individual member. Thus, the second target communication may include modifications to offset the decrease in the associated levels of the target communication attribute such as recommending a different tool or modifications to the tool.

At Block 318, a step of transmitting the second target communication to the individual member is provided. The step of Block 318 is the same as or similar to the step of Block 312.

The method described in FIG. 3 can include more or less steps. One or more steps or any combination of steps may also be repeated in the method described in FIG. 3. For example, additional target communications may be generated based on additional analysis of plurality of communications from any number of periods of time. In such examples, steps of the Blocks 308, 310, and 312 may be repeated.

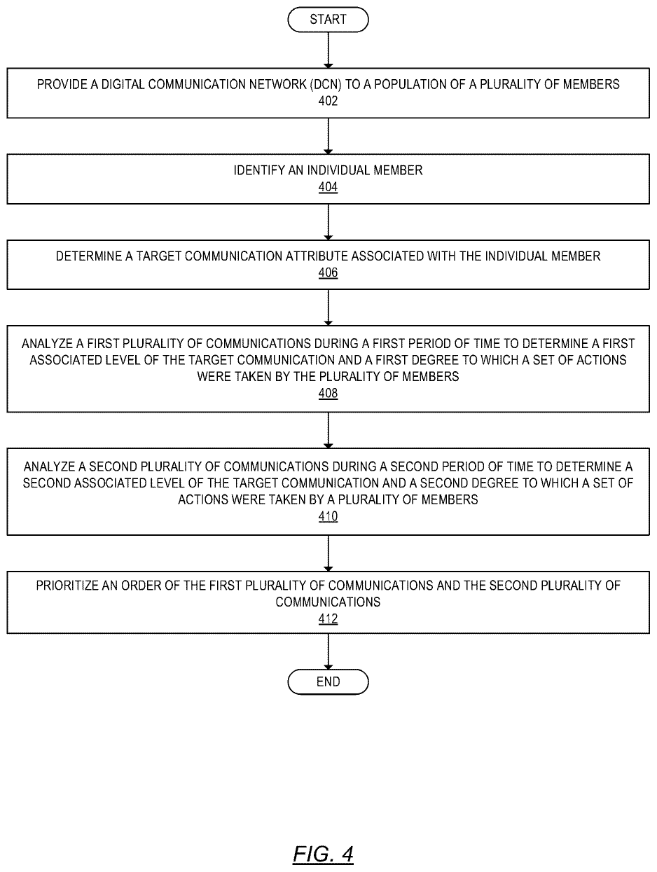

FIG. 4 is a logic flow diagram that illustrates a method, and a result of execution of computer program instructions, in accordance with various embodiments. The method can be used to select and prioritize an order of a subset of communications based on a target communication attribute of an individual member and a degree to which members of the DCN have taken one or more actions. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 402, a step of providing a digital communication network (DCN) to a population of a plurality of members is provided. The step of Block 402 may be the same as or similar to the step of Block 202 of FIG. 2 described above.

At Block 404, a step of identifying an individual member is provided. The step of Block 404 may be the same as or similar to the step of Block 204 of FIG. 2 described above.

At Block 406, a step of determining a target communication attribute associated with the individual member is provided. The step of Block 406 may be the same as or similar to the step of Block 206 of FIG. 2 described above.

At Block 408, a step of analyzing a first plurality of communications during a first period of time to determine a first associated level of the target communication attribute and a first degree to which a set of actions were taken by the plurality of members is provided. The step of Block 408 is the same as or similar to the step of Block 208, except that the step of Block 408 also includes determining the first degree to which a set of actions were taken by the plurality of members. The set of actions may be the same as or similar to the set of actions (108) and the degree may be the same as or similar to the degree (110).

As previously described, the degree corresponds to how much a member has participated in or performed the action(s). For example, a member who participates in yoga weekly will have a higher degree of the action yoga than a member who participates in yoga once a month. The degree can be determined by searching for key words in the plurality of communications. In other examples, the degree can be determined based on data from a wearable monitor such as the wearable monitor (156). In still other examples, the degree can be based on member input received via a user device such as the user device (150).

At Block 410, a step of analyzing a second plurality of communications during a second period of time to determine a second associated level of the target communication attribute and a second degree to which a set of actions were taken by the plurality of members is provided. The step of Block 414 is the same as or similar to the step of Block 408, except that the second period of time occurs after the first period of time begins.

At Block 412, a step of prioritizing an order of communications from the first plurality of communications and the second plurality of communications is provided. The order of communications may be prioritized based on, for example, the first associated level of the target communication attribute, the second associated level of the target communication attribute, the first degree, and the second degree. For example, communications with higher associated levels of the target communication attribute and higher levels of the degree to which the plurality of members have taken action may be prioritized higher than communications with lower associated levels of the target communication attribute and lower levels of the degree. By prioritizing communications with high levels of degree and high associated levels of the target communication attribute, the individual member may be more receptive to such communications and the communications may incentivize the individual member to perform, or continue to perform, the action.

The method described in FIG. 4 can include more or less steps. One or more steps or any combination of steps may also be repeated in the method described in FIG. 3.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 5A, the computing system (500) may include one or more computer processor(s) (502), non-persistent storage device(s) (504), persistent storage device(s) (506), a communication interface (508) (e.g., Bluetooth interface, infrared interface, network interface, optical interface, etc.), and numerous other elements and functionalities that implement the features and elements of the disclosure. The computer processor(s) (502) may be an integrated circuit for processing instructions. The computer processor(s) (502) may be one or more cores, or micro-cores, of a processor. The computer processor(s) (502) includes one or more processors. The computer processor(s) (502) may include a central processing unit (CPU), a graphics processing unit (GPU), a tensor processing unit (TPU), combinations thereof, etc.

The input device(s) (510) may include a touchscreen, keyboard, mouse, microphone, touchpad, electronic pen, or any other type of input device. The input device(s) (510) may receive inputs from a user that are responsive to data and messages presented by the output device(s) (512). The inputs may include text input, audio input, video input, etc., which may be processed and transmitted by the computing system (500) in accordance with one or more embodiments. The communication interface (508) may include an integrated circuit for connecting the computing system (500) to a network (not shown) (e.g., a local area network (LAN), a wide area network (WAN) such as the Internet, mobile network, or any other type of network) or to another device, such as another computing device, and combinations thereof.

Further, the output device(s) (512) may include a display device, a printer, external storage, or any other output device. One or more of the output device(s) (512) may be the same or different from the input device(s) (510). The input device(s) (510) and output device(s) (512) may be locally or remotely connected to the computer processor(s) (502). Many different types of computing systems exist, and the aforementioned input device(s) (510) and output device(s) (512) may take other forms. The output device(s) (512) may display data and messages that are transmitted and received by the computing system (500). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (502), is configured to perform one or more embodiments, which may include transmitting, receiving, presenting, and displaying data and messages described in the other figures of the disclosure.

The computing system (500) in FIG. 5A may be connected to, or be a part of, a network. For example, as shown in FIG. 5B, the network (520) may include multiple nodes (e.g., node X (522) and node Y (524), as well as extant intervening nodes between node X (522) and node Y (524)). Each node may correspond to a computing system, such as the computing system shown in FIG. 5A, or a group of nodes combined may correspond to the computing system shown in FIG. 5A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (500) may be located at a remote location and connected to the other elements over a network.

The nodes (e.g., node X (522) and node Y (524)) in the network (520) may be configured to provide services for a client device (526). The services may include receiving requests and transmitting responses to the client device (526). For example, the nodes may be part of a cloud computing system. The client device (526) may be a computing system, such as the computing system shown in FIG. 5A. Further, the client device (526) may include or perform all or a portion of one or more embodiments.

The computing system of FIG. 5A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

The present invention may be further exemplified by one, or a combination of one or more of, the following statements:
Statement 1. A method to slow progression of an adverse health condition in an individual member of a population, the method comprising: providing a digital communication network (DCN) for members of the population to communicate with each other; selecting a plurality of messages from members of the population to show to the individual member; analyzing the plurality of messages to determine, for each message, an associated level of at least one communication attribute, wherein the at least one communication attribute comprises at least one of: empathy, attachment, introspection, and agency; prioritizing an order in which the individual member sees the plurality of messages based on the associated level of at least one communication attribute for each message; and presenting the plurality of messages to the individual member in the order, wherein messages with higher levels of the at least one communication attribute are shown first.
Statement 2. The method of Statement 1, further comprising automatically analyzing messages in the DCN to determine, for each message, an associated level of at least one communication attribute.
Statement 3. The method of Statement 1, wherein the adverse health condition comprises at least one of: chronic systemic inflammation, malaise, and low energy.
Statement 4. The method of Statement 1, wherein the adverse health condition comprises a disease.
Statement 5. The method of Statement 4, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.
Statement 6. The method of Statement 1, wherein the adverse health condition comprises a prodromal version of a disease.
Statement 7. The method of Statement 1, wherein the adverse health condition comprises a health risk.
Statement 8. The method of Statement 7, wherein the health risk comprises at least one of: a risk of falling, and a risk of becoming infected.
Statement 9. The method of Statement 1, wherein the adverse health condition comprises social dysfunction.
Statement 10. The method of Statement 1, wherein communications in the DCN comprise at least one of: one-to-one communications, one-to-many communications, one-to-system communications, system-to-one communications, and system-to-many communications.
Statement 11. The method of Statement 1, wherein the DCN comprises an artificial intelligence (AI) bot configured to derive messages based on analysis of communication in the DCN or biometrics provided to the DCN.
Statement 12. A method to slow progression of an adverse health condition in an individual member of a population, the method comprising: providing a digital communication network (DCN) for members of the population to communicate with each other; analyzing a first plurality of messages in the DCN from a first period of time to determine a first associated level of at least one communication attribute, wherein the at least one communication attribute comprises at least one of: empathy, attachment, introspection, and agency; sending a communication to the individual member offering a tool associated with living a healthier lifestyle through communications on the digital communications network during a second period of time; analyzing a second plurality of messages in the DCN from the second period of time to determine a second associated level of the at least one communication attribute; and modifying the tools based on a difference in the first associated level of the at least one communication attribute and the second associated level of the at least one communication attribute.
Statement 13. The method of Statement 12, wherein the first period of time and the second period of time are one of: one day, one week, and one month.
Statement 14. The method of Statement 12, wherein the tool is a wearable device configured to measure a biomarker.
Statement 15. The method of Statement 12, wherein the tool is kits to measure a biomarker at home.
Statement 16. The method of Statement 15, wherein the biomarker is one of: glucose, lactate, ketones, C-reactive proteins (CRP), a cytokine, an antibody, steps, heart rate (HR), heart rate variability (HRV), blood oxygen levels, breathing rate, and a wheezing rate.
Statement 17. The method of Statement 15, wherein the biomarker is determined from a blood sample, a saliva sample, a stool sample, and/or a breath sample.
Statement 18. The method of Statement 12, wherein the tool is a health-inducing game.
Statement 19. The method of Statement 12, wherein the tool is a psychometric instrument.
Statement 20. The method of Statement 12, wherein the tool is a health-improving program configured to address at least one of: stress, anxiety, depression, happiness, social connectiveness.
Statement 21. The method of Statement 12, further comprising automatically analyzing messages in the DCN to determine, for each message, an associated level of at least one communication attribute.
Statement 22. The method of Statement 12, further comprising repeatedly analyzing messages in the DCN and modifying the tools based on differences in levels of the at least one communication attribute.
Statement 23. The method of Statement 12, wherein the adverse health condition comprises at least one of: chronic systemic inflammation, malaise, and low energy.
Statement 24. The method of Statement 12, wherein the adverse health condition comprises a disease.
Statement 25. The method of Statement 24, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.
Statement 26. The method of Statement 12, wherein the adverse health condition comprises a prodromal version of a disease.
Statement 27. The method of Statement 12, wherein the adverse health condition comprises a health risk.
Statement 28. The method of Statement 27, wherein the health risk comprises at least one of: a risk of falling, and a risk of becoming infected.
Statement 29. The method of Statement 12, wherein the adverse health condition comprises social dysfunction.
Statement 30. The method of Statement 12, wherein communications in the DCN comprise at least one of: one-to-one communications, one-to-many communications, one-to-system communications, system-to-one communications, and system-to-many communications.
Statement 31. The method of Statement 12, wherein the DCN comprises an artificial intelligence (AI) bot configured to derive messages based on analysis of communication in the DCN or biometrics provided to the DCN.
Statement 32. A method to slow progression of an adverse health condition in an individual member of a population, the method comprising: providing a digital communication network (DCN) for members of the population to communicate with each other; analyzing a first plurality of messages in the DCN from a first period of time to determine a first associated level of at least one communication attribute, wherein the at least one communication attribute comprises at least one of: empathy, attachment, introspection, and agency; sending to the individual member a standard communication offering a tool associated with living a healthier lifestyle through communications on the digital communications network during a second period of time; analyzing a second plurality of messages in the DCN from the second period of time to determine a second associated level of the at least one communication attribute; and modifying the standard communication offering the tool based on a difference in the first associated level of the at least one communication attribute and the second associated level of the at least one communication attribute.
Statement 33. The method of Statement 32, wherein the first period of time and the second period of time are one of: one day, one week, and one month.
Statement 34. The method of Statement 32, wherein the tool is a wearable device configured to measure a biomarker.
Statement 35. The method of Statement 32, wherein the tool is kits to measure a biomarker at home.
Statement 36. The method of Statement 35, wherein the biomarker is one of: glucose, lactate, ketones, C-reactive proteins (CRP), a cytokine, an antibody, steps, heart rate (HR), heart rate variability (HRV), blood oxygen levels, breathing rate, and a wheezing rate.
Statement 37. The method of Statement 35, wherein the biomarker is determined from a blood sample, a saliva sample, a stool sample, and/or a breath sample.
Statement 38. The method of Statement 32, wherein the tool is a health-inducing game.
Statement 39. The method of Statement 32, wherein the tool is a psychometric instrument.
Statement 40. The method of Statement 32, wherein the tool is a health-improving program configured to address at least one of: stress, anxiety, depression, happiness, social connectiveness.
Statement 41. The method of Statement 32, further comprising automatically analyzing messages in the DCN to determine, for each message, an associated level of at least one communication attribute.
Statement 42. The method of Statement 32, further comprising repeatedly analyzing messages in the DCN and modifying the standard communication based on differences in levels of the at least one communication attribute.
Statement 43. The method of Statement 32, wherein the adverse health condition comprises at least one of: chronic systemic inflammation, malaise, and low energy.
Statement 44. The method of Statement 32, wherein the adverse health condition comprises a disease.
Statement 45. The method of Statement 44, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.
Statement 46. The method of Statement 32, wherein the adverse health condition comprises a prodromal version of a disease.
Statement 47. The method of Statement 32, wherein the adverse health condition comprises a health risk.
Statement 48. The method of Statement 47, wherein the health risk comprises at least one of: a risk of falling, and a risk of becoming infected.
Statement 49. The method of Statement 32, wherein the adverse health condition comprises social dysfunction.
Statement 50. The method of Statement 32, wherein communications in the DCN comprise at least one of: one-to-one communications, one-to-many communications, one-to-system communications, system-to-one communications, and system-to-many communications.
Statement 51. The method of Statement 32, wherein the DCN comprises an artificial intelligence (AI) bot configured to derive messages based on analysis of communication in the DCN or biometrics provided to the DCN.
Statement 52. A method to slow progression of an adverse health condition in an individual member of a population, the method comprising: providing a digital communication network (DCN) for members of the population to communicate with each other; analyzing a first plurality of messages over a first period of time to determine a first associated level of at least one communication attribute, wherein the at least one communication attribute comprises at least one of: empathy, attachment, introspection, and agency; generating a plurality of summaries of communications on the DCN, each summary describing communications which are similar to each other; providing the summaries to the members of the population during a second period of time; analyzing a second plurality of messages over the second period of time to determine a second associated level of at least one communication attribute; and modifying the plurality of summaries based on a difference in the first associated level of the at least one communication attribute and the second associated level of the at least one communication attribute.
Statement 53. The method of Statement 52, wherein the summaries are automatically generated by the DCN.
Statement 54. The method of Statement 52, further comprising tailoring the summaries for individual members of the population.
Statement 55. The method of Statement 54, wherein different summaries are provided to different members.
Statement 56. The method of Statement 52, wherein the first period of time and the second period of time are one of: one day, one week, and one month.
Statement 57. The method of Statement 52, further comprising automatically analyzing messages in the DCN to determine, for each message, an associated level of at least one communication attribute.
Statement 58. The method of Statement 52, wherein the adverse health condition comprises at least one of: chronic systemic inflammation, malaise, and low energy.
Statement 59. The method of Statement 52, wherein the adverse health condition comprises a disease.
Statement 60. The method of Statement 59, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.
Statement 61. The method of Statement 52, wherein the adverse health condition comprises a prodromal version of a disease.
Statement 62. The method of Statement 52, wherein the adverse health condition comprises a health risk.
Statement 63. The method of Statement 62, wherein the health risk comprises at least one of: a risk of falling, and a risk of becoming infected.
Statement 64. The method of Statement 52, wherein the adverse health condition comprises social dysfunction.
Statement 65. The method of Statement 52, wherein communications in the DCN comprise at least one of: one-to-one communications, one-to-many communications, one-to-system communications, system-to-one communications, and system-to-many communications.
Statement 66. The method of Statement 52, wherein the DCN comprises an artificial intelligence (AI) bot configured to derive messages based on analysis of communication in the DCN or biometrics provided to the DCN.
Statement 67. A method to slow progression of an adverse health condition in an individual member of a population, the method comprising: providing a digital communication network (DCN) for members of the population to communicate with each other; selecting a first plurality of messages from members of the population to show to the individual member; prioritizing an order in which the individual member sees the first plurality of messages based on personalized criteria for the individual member; presenting the first plurality of messages in order to the individual member; analyzing communications in the DCN over a first period of time to determine a first associated level of at least one communication attribute, wherein the at least one communication attribute comprises at least one of: empathy, attachment, introspection, and agency; analyzing communications in the DCN over a second period of time to determine a second associated level of at least one communication attribute; and modifying the personalized criteria to generate updated personalized criteria based on a difference in the first associated level of the at least one communication attribute and the second associated level of the at least one communication attribute.
Statement 68. The method of Statement 67, wherein the first period of time and the second period of time are one of: one day, one week, and one month.
Statement 69. The method of Statement 67, further comprising automatically analyzing messages in the DCN to determine, for each message, an associated level of at least one communication attribute.
Statement 70. The method of Statement 67, wherein the adverse health condition comprises at least one of: chronic systemic inflammation, malaise, and low energy.
Statement 71. The method of Statement 67, wherein the adverse health condition comprises a disease.
Statement 72. The method of Statement 71, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.
Statement 73. The method of Statement 67, wherein the adverse health condition comprises a prodromal version of a disease.
Statement 74. The method of Statement 67, wherein the adverse health condition comprises a health risk.
Statement 75. The method of Statement 74, wherein the health risk comprises at least one of: a risk of falling, and a risk of becoming infected.
Statement 76. The method of Statement 67, wherein the adverse health condition comprises social dysfunction.
Statement 77. The method of Statement 67, wherein communications in the DCN comprise at least one of: one-to-one communications, one-to-many communications, one-to-system communications, system-to-one communications, and system-to-many communications.
Statement 78. The method of Statement 67, wherein the DCN comprises an artificial intelligence (AI) bot configured to derive messages based on analysis of communication in the DCN or biometrics provided to the DCN.
Statement 79. The method of Statement 67, further comprising repeatedly analyzing communications in the DCN and modifying the personalized criteria.
Statement 80. The method of Statement 67, further comprising analyzing communications in the DCN to determine the adverse health condition.
Statement 81. The method of Statement 67, further comprising analyzing communication in the DCN to generate an ontology of health conditions for the individual member, wherein the ontology of health conditions comprises the adverse health condition.
Statement 82. A method to slow progression of an adverse health condition in an individual member of a population, the method comprising: providing a digital communication network (DCN) for members of the population to communicate with each other; determining a first degree to which a given set of actions were adopted by the members of the population during a first period; analyzing communications in the DCN over the first period of time to determine a first associated level of at least one communication attribute, wherein the at least one communication attribute comprises at least one of: empathy, attachment, introspection, and agency; determining a second degree to which a given set of actions were adopted by the members of the population during a second period; analyzing communications in the DCN over the second period of time to determine a second associated level of the at least one communication attribute; selecting a first plurality of messages from members of the population to show to the individual member; and prioritizing an order in which the individual member sees the first plurality of messages based on personalized criteria for the individual member, wherein the personalized criteria are based, at least in part, on the first associated level and the second associated level.
Statement 83. The method of Statement 82, wherein the personalized criteria are selected to increase probability of adoption by the individual member of an action from the given set of actions.
Statement 84. The method of Statement 82, wherein the personalized criteria are selected to increase probability of the individual member maintaining the action.
Statement 85. The method of Statement 82, wherein the first period of time and the second period of time are one of: one day, one week, and one month.
Statement 86. The method of Statement 82, wherein the first period of time and the second period of time are determined by a flow of messages in the DCN.
Statement 87. The method of Statement 82, wherein the given set of actions comprise at least one of: lifestyle changes, medical interventions, changes in social interactions, and changes in a living environment.
Statement 88. The method of Statement 82, further comprising automatically analyzing messages in the DCN to determine an associated level of the at least one communication attribute.
Statement 89. The method of Statement 82, wherein the adverse health condition comprises at least one of: chronic systemic inflammation, malaise, and low energy.
Statement 90. The method of Statement 82, wherein the adverse health condition comprises a disease.
Statement 91. The method of Statement 90, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.
Statement 92. The method of Statement 82, wherein the adverse health condition comprises a prodromal version of a disease.
Statement 93. The method of Statement 82, wherein the adverse health condition comprises a health risk.
Statement 94. The method of Statement 93, wherein the health risk comprises at least one of: a risk of falling, and a risk of becoming infected.
Statement 95. The method of Statement 82, wherein the adverse health condition comprises social dysfunction.
Statement 96. The method of Statement 82, wherein communications in the DCN comprise at least one of: one-to-one communications, one-to-many communications, one-to-system communications, system-to-one communications, and system-to-many communications.
Statement 97. The method of Statement 82, wherein the DCN comprises an artificial intelligence (AI) bot configured to derive messages based on analysis of communication in the DCN or biometrics provided to the DCN.
Statement 98. The method of Statement 82, further comprising repeatedly analyzing communications in the DCN and modifying the personalized criteria.
Statement 99. The method of Statement 82, further comprising analyzing communications in the DCN to determine the adverse health condition.
Statement 100. The method of Statement 82, further comprising analyzing communication in the DCN to generate an ontology of health conditions for the individual member, wherein the ontology of health conditions comprises the adverse health condition.
Statement 101. The method of Statement 1, wherein the method is used to reduce health care costs in the population.
Statement 102. The method of Statement 1, wherein the method is used to reduce health care risk in the population.
Statement 103. The method of Statement 1, wherein the method is used to manage a disease in at least one of the population and one or more members of the population.
Statement 101. The method of Statement 1, wherein the method is used to reduce health care costs in the population.
Statement 102. The method of Statement 1, wherein the method is used to reduce health care risk in the population.
Statement 103. The method of Statement 1, wherein the method is used to manage a disease in at least one of the population and one or more members of the population.
Statement 101. The method of Statement 1, wherein the method is used to reduce health care costs in the population.
Statement 102. The method of Statement 1, wherein the method is used to reduce health care risk in the population.
Statement 103. The method of Statement 1, wherein the method is used to manage a disease in at least one of the population and one or more members of the population.
Statement 104. The method of Statement 12, wherein the method is used to reduce health care costs in the population.
Statement 105. The method of Statement 12, wherein the method is used to reduce health care risk in the population.
Statement 106. The method of Statement 12, wherein the method is used to manage a disease in at least one of the population and one or more members of the population.
Statement 107. The method of Statement 32, wherein the method is used to reduce health care costs in the population.
Statement 108. The method of Statement 32, wherein the method is used to reduce health care risk in the population.
Statement 109. The method of Statement 32, wherein the method is used to manage a disease in at least one of the population and one or more members of the population.
Statement 110. The method of Statement 52, wherein the method is used to reduce health care costs in the population.
Statement 111. The method of Statement 52, wherein the method is used to reduce health care risk in the population.
Statement 112. The method of Statement 52, wherein the method is used to manage a disease in at least one of the population and one or more members of the population.
Statement 113. The method of Statement 67, wherein the method is used to reduce health care costs in the population.
Statement 114. The method of Statement 67, wherein the method is used to reduce health care risk in the population.
Statement 115. The method of Statement 67, wherein the method is used to manage a disease in at least one of the population and one or more members of the population.
Statement 116. The method of Statement 82, wherein the method is used to reduce health care costs in the population.
Statement 117. The method of Statement 82, wherein the method is used to reduce health care risk in the population.
Statement 118. The method of Statement 82, wherein the method is used to manage a disease in at least one of the population and one or more members of the population.

What is claimed is:

1. A method comprising:
providing a digital communication network (DCN) to a plurality of members of a population to communicate with each other and the DCN;
identifying an individual member from the plurality of members in the DCN;
determining a target communication attribute associated with the individual member, wherein the target communication attribute corresponds to a communication attribute valued by the individual member;
analyzing a plurality of communications from the plurality of members to determine, for each communication, an associated level of the target communication attribute; and
prioritizing an order of the plurality of communications based on the associated level of the target communication attribute, wherein communications with higher associate levels of the target communication attribute are prioritized higher than communications with lower or no associated levels of the target communication attribute.

2. The method of claim 1, wherein the target communication attribute comprises one or more of: empathy, attachment, introspection, and agency.

3. The method of claim 1, wherein the plurality of communications are a first plurality of communications analyzed during a first period of time and the associated level of the target communication attribute is a first associated level of the target communication attribute, and wherein the method further comprises:
generating a first target communication to the individual member based on the target communication attribute;
transmitting the first target communication to the individual member during a second period of time, the second period of time occurring after the first period of time;
analyzing a second plurality of communications during the second period of time to determine a second associated level of the target communication attribute;
generating a second target communication to the individual member based on a difference between the first associated level of the target communication attribute and the second associated level of the target communication attribute; and
transmitting the second target communication to the individual member.

4. The method of claim 3, wherein the first period of time and the second period of time are one of: one or more days, one or more weeks, and one or more months.

5. The method of claim 3, wherein the first target communication includes a plurality of summaries, each summary describing a subset of communications from the plurality of communications that are similar to each other, and wherein the second target communication includes a modified plurality of summaries.

6. The method of claim 5, wherein different summaries of the plurality of summaries are transmitted to different members of the plurality of members.

7. The method of claim 3, wherein the first target communication includes a first recommendation for a tool associated with living a healthy lifestyle and the second target communication includes a second recommendation for an adjustment to the tool or a different tool.

8. The method of claim 7, wherein the tool comprises at least one of a wearable device configured to measure a biomarker, a kit to measure a biomarker, a health-inducing game, a psychometric instrument, and a health-improving program.

9. The method of claim 1, wherein the method is used to slow progression of an adverse health condition of the individual member.

10. The method of claim 9, wherein the adverse condition is determined by automatically analyzing communications from the individual member in the DCN.

11. The method of claim 10, wherein the adverse condition comprises at least one of chronic systemic inflammation, malaise, low energy, a disease, a health risk, social dysfunction, or a prodromal disease.

12. The method of claim 11, wherein the disease comprises at least one of obesity, diabetes, rheumatoid arthritis, Crohn's disease, psoriasis, eczema, cardiovascular disease, congestive heart failure, chronic obstructive pulmonary disease, asthma, depression, or anxiety.

13. The method of claim 11, wherein the health risk is at least one of falling or becoming infected with an illness.

14. A method comprising:
providing a digital communication network (DCN) to a plurality of members of a population to communicate with each other and the DCN;
identifying an individual member from the plurality of members in the DCN;
analyzing a first plurality of communications from the plurality of members over a first period of time to determine:
a first degree to which a set of actions were taken by the plurality of members during the first period of time, and
a first associated level of a communication attribute;
analyzing a second plurality of communications from the plurality of members over a second period of time to determine:
a second degree to which the set of actions were taken by the plurality of members during the second period of time, and
a second associated level of the communication attribute;
prioritizing an order of the subset of communications based on the first associated level of the communication attribute, the second associated level of the communication attribute, the first degree, and the second degree.

15. The method of claim 14, wherein the order of the subset of communications is further prioritized to increase a probability of adoption by the individual member of an action from the set of actions.

16. The method of claim 15, wherein the order of the subset of communications is further prioritized to increase a probability of the individual member maintaining the adopted action.

17. The method of claim 14, wherein the set of actions comprises at least one of: lifestyle changes, medical interventions, changes in social interactions, and changes in a living environment.

18. The method of claim 14, wherein a difference between the first degree and the second degree meeting the predetermined threshold correlates to at least one of an increased degree or a decreased degree of the set of actions taken by the plurality of members.

19. The method of claim 14, wherein the method is used to slow progression of an adverse health condition of the individual member.

20. A system comprising:
a computer processor;
a data repository in communication with the computer processor and storing:
a plurality of communications,
a communication attribute, and
an associated level of a communication attribute;
a digital communications network (DCN) which, when executed by the computer processor, provides a network for a plurality of members of a population to interact with at least each other or the DCN; and
a server controller which, when executed by the computer processor:
identifies an individual member from the plurality of members in the DCN;
determining a target communication attribute associated with the individual member, wherein the target communication attribute corresponds to a communication attribute valued by the individual member;
identifies a plurality of communications from the plurality of members;
analyzes the plurality of communications from the plurality of members to determine, for each communication, an associated level of the target communication attribute; and
prioritizes an order of the plurality of communications based on the associated level of the target communication attribute, wherein communications with higher associate levels of the target communication attribute are prioritized higher than communications with lower or no associated levels of the target communication attribute.
